(19) United States
(12) Patent Application Publication
(10) Pub. No.: US 2024/0277942 A1
(43) Pub. Date: Aug. 22, 2024

(54) ELECTROMECHANICAL ARCHITECTURE FOR CONNECTED PREFILLED DISPOSABLE SYRINGES

(71) Applicant: Flextronics AP, LLC, San Jose, CA (US)

(72) Inventor: Marco De Angeli, Barzana (IT)

(73) Assignee: Flextronics AP, LLC, San Jose, CA (US)

(21) Appl. No.: 18/581,967

(22) Filed: Feb. 20, 2024

Related U.S. Application Data
(60) Provisional application No. 63/447,237, filed on Feb. 21, 2023.

(51) Int. Cl. A61M 5/315

(52) U.S. Cl. CPC A61M 5/3157; A61M 5/31591; A61M 2205/3317; A61M 2205/3561; A61M 2205/3584; A61M 2205/50; A61M 2205/8206

(57) ABSTRACT

A smart syringe includes a barrel and a plunger axially translatable within the barrel. The plunger includes a first body portion coupled to a second body portion. A printed circuit board is coupled and positioned between the first body portion and the second body portion. The printed circuit board can include a ground contact disposed on a first lateral side of the printed circuit, a plurality of contact pads disposed on a second lateral side of the printed circuit board, and a microcontroller coupled to a surface of the printed circuit board. A conductive ring is disposed about an outer circumference of the plunger. The conductive ring includes sliding contacts configured to engage the ground contact and the plurality of contact pads of the printed circuit board. The microcontroller is configured to detect a change in conductivity to identify a position of the plunger relative to the barrel.

### CROSS REFERENCE TO RELATED APPLICATIONS

This application claims the benefit of U.S. Provisional Application No. 63/447,237 filed on Feb. 21, 2023, which is incorporated by reference as if fully set forth.

### TECHNICAL FIELD

The present disclosure is directed to disposable syringes, and more particularly it is directed to prefilled disposable syringes with an electromechanical architecture for data collection and transfer.

### BACKGROUND OF THE INVENTION

As is readily known, a syringe is a pumping device generally including a plunger disposed tightly within a cylindrical tube or barrel, and syringes are generally used in the medical industry to inject and dispense a fluid, such as liquid medication. Recently, there is a desire to obtain more data related to medical procedures to improve clinical trials and the overall health of patients. Therefore, smart syringes have been developed which are utilized to collect injection information, provide more refined insights, and perform better statistical analysis of medical trial outcomes. For example, traditional smart syringes can be configured to record one or more of the following: time of injection, duration of injection, dose administered, drug name, expiry date, and other similar data. An issue arises when introducing the smart syringe technology into disposable syringes due to the higher costs associated with smart syringes and the disposable nature of disposable syringes, resulting in an expensive disposable product.

Therefore, there is a need for a cost effective disposable syringe that utilizes traditional smart syringe technology, as well as includes further capabilities to collect data that has not previously been collected using smart syringe technology.

### SUMMARY OF THE INVENTION

According to one aspect, a syringe (i.e., a “smart syringe”) is disclosed according to the present disclosure. The syringe can include a barrel including a barrel flange positioned at an axial end of the barrel. A plunger can be axially translatable within the barrel along a central axis of the plunger, and the plunger can include a first body portion coupled to a second body portion. A printed circuit board can be coupled and positioned between the first body portion and the second body portion. The printed circuit board can include a ground contact disposed on a first lateral side of the printed circuit, a plurality of contact pads disposed on a second lateral side of the printed circuit board, and a microcontroller coupled to a surface of the printed circuit board. A conductive ring can be disposed about an outer circumference of the plunger, the conductive ring can include sliding contacts configured to engage the ground contact and the plurality of contact pads of the printed circuit board. The microcontroller can be configured to detect a change in conductivity to identify a position of the plunger relative to the barrel.

In one aspect, the first body portion and the second body portion are coupled together through a snap-fit connection.

In one aspect, the first body portion and the second body portion are a first half portion and a second half portion of the plunger, respectively.

In one aspect, the second body portion includes a pin positioned on a radially inner surface of the second body portion, relative to the central axis of the plunger.

In one aspect, the printed circuit board includes an aperture extending through the printed circuit board, the aperture being configured to accept the pin of the second body portion for positioning of the printed circuit board relative to the second body portion.

In one aspect, the printed circuit board includes a battery connector disposed at an end of the printed circuit board, the battery connector being configured for accepting and securing a battery of the syringe.

In one aspect, the battery is a coin cell battery.

In one aspect, the ground contact of the printed circuit board is a continuous ground contact that extends from a first end of the printed circuit board to a second end of the printed circuit board.

In one aspect, the plurality of contact pads are a discontinuous group of individual contact pads that extend from a first end of the printed circuit board to a second end of the printed circuit board.

In one aspect, the discontinuous group of individual contact pads includes a nonconductive space positioned between each adjacent contact pad of the plurality of contact pads.

In one aspect, the microcontroller is a Bluetooth low energy chip that is configured to wirelessly transmit data to a device remote from the syringe.

In one aspect, the surface of the printed circuit board the microcontroller is coupled to is perpendicular to the first lateral side and the second lateral side of the printed circuit board.

In one aspect, the first lateral side of the printed circuit board is parallel with the second lateral side of the printed circuit board.

In one aspect, the syringe is a pre-filled disposable syringe.

In one aspect, the conductive ring includes a circular shaped conductive ring body and a first sliding contact and a second sliding contact each extending axially away from the conductive ring body.

In one aspect, the first sliding contact is configured to engage the ground contact of the printed circuit board, and the second sliding contact is configured to engage the plurality of contact pads of the printed circuit board.

In one aspect, the conductive ring includes an inner diameter greater than an outer diameter of a portion of the plunger configured to axially translate into the barrel.

In one aspect, the conductive ring is axially translatable along an outer surface of the plunger during an axial translation of the plunger into the barrel.

In one aspect, during the axial translation of the plunger into the barrel the conductive ring contacts the barrel flange, preventing axial translation of the conductive ring relative to the barrel.

In one aspect, before the axial translation of the plunger into the barrel, the conductive ring is axially offset from the barrel flange.

### BRIEF DESCRIPTION OF THE DRAWINGS

The foregoing Summary as well as the following Detailed Description will be best understood when read in conjunction with the appended drawings, which illustrate a preferred embodiment of the disclosure. In the drawings:

FIG. 1 is a perspective view of a syringe according to the present disclosure.

FIG. 2 is a side view of a plunger of the syringe of FIG. 1.

FIG. 3 is an exploded perspective view of the plunger of FIG. 2.

FIG. 4 is a perspective view of a printed circuit board of the plunger of FIG. 2.

FIG. 5 is a perspective view of a conductive ring of the syringe of FIG. 1.

FIG. 6A is a perspective view of the syringe of FIG. 1 in a first position.

FIG. 6B is a perspective view of the syringe of FIG. 1 in a second position.

FIG. 6C is a perspective view of the syringe of FIG. 1 in a third position.

### DETAILED DESCRIPTION OF THE PREFERRED EMBODIMENTS

Certain terminology is used in the following description for convenience only and is not limiting. The words “front”, “rear”, “upper”, and “lower” designate directions in the drawings to which reference is made. The words “inwardly” and “outwardly” refer to directions towards and away from parts referenced in the drawings. “Axially” refers to a direction along the axis of a shaft, pin, tube, barrel, rod, or the like. A reference to a list of items that are cited as “at least one of a, b, or c” (where a, b, and c represent the items being listed) means any single one of the items a, b, or c, or combinations thereof are included. The terminology includes the words specifically noted above, derivatives thereof, and words of similar import.

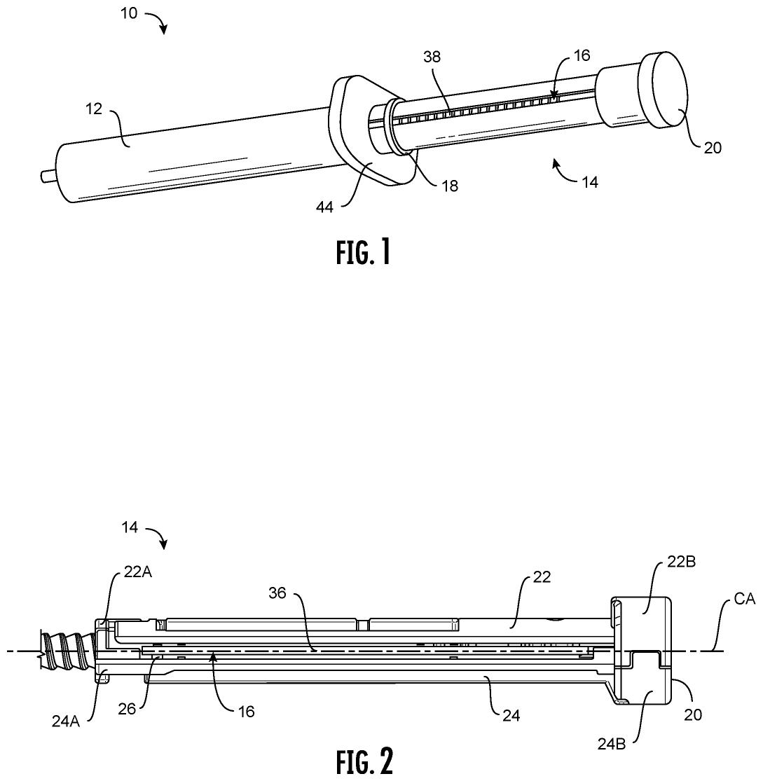

FIG. 1 is a perspective view of a syringe 10 according to the present disclosure. The syringe 10 is a pumping device which is generally used in the medical industry to inject and dispense a fluid such as liquid medication, among other options not specifically listed. In some examples, the syringe 10 can be a pre-filled disposable syringe, such that a user receives the syringe pre-filled with a fluid, injects the fluid, and then disposes of (throws away) the pre-filled disposable syringe. In other examples, the syringe 10 may not be a pre-filled or disposable syringe.

The syringe 10 can include a barrel 12, a plunger 14, a printed circuit board 16 within the plunger 14, a conductive ring 18, and a needle (not shown). The barrel 12 is the body of the syringe 10 that houses the plunger 14 and the fluid to be dispensed by the syringe 10. In some examples, as illustrated, the barrel 12 can have a generally elongated cylindrical shape. In other examples, the barrel 12 can have a shape other than an elongated cylindrical shape. Disposed at the end of the barrel 12, the needle can be coupled to the barrel 12. As is known, the needle is configured to be inserted into the user when dispensing the fluid or medication from within the syringe 10. The plunger 14 is disposed tightly within the barrel 12. Specifically, the plunger 14 is axially aligned with the barrel 12, and the plunger 14 is axially translatable within the barrel 12 along a central axis CA of the plunger 14. When pressed and/or squeezed, an end of the plunger 14 (not illustrated in FIG. 1) is configured to force the fluid within the barrel 12 out through the needle (not shown). The plunger 14 includes a plunger end 20 extending outside the barrel 12, which a user presses or squeezes to force the plunger 14 to axially translate into the barrel 12 to force the fluid within the barrel 12 out through the needle, as is readily known.

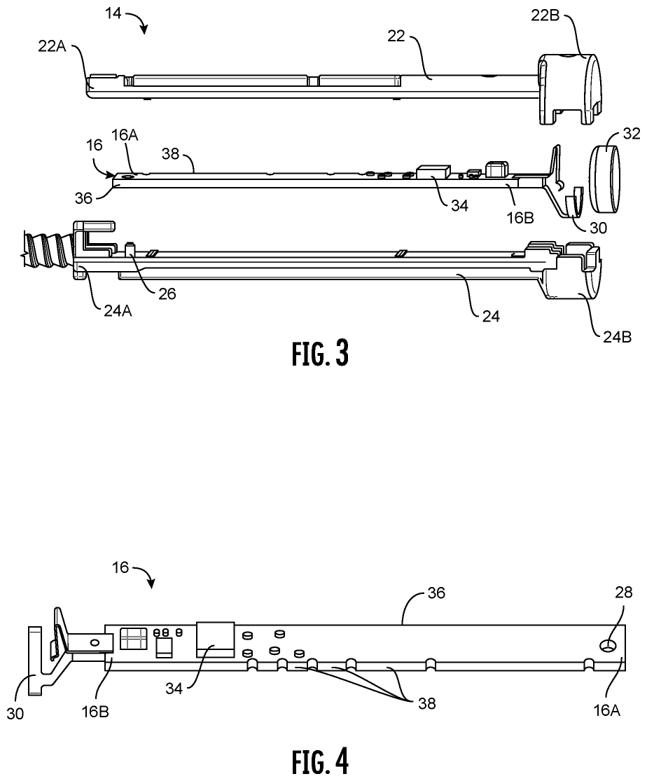

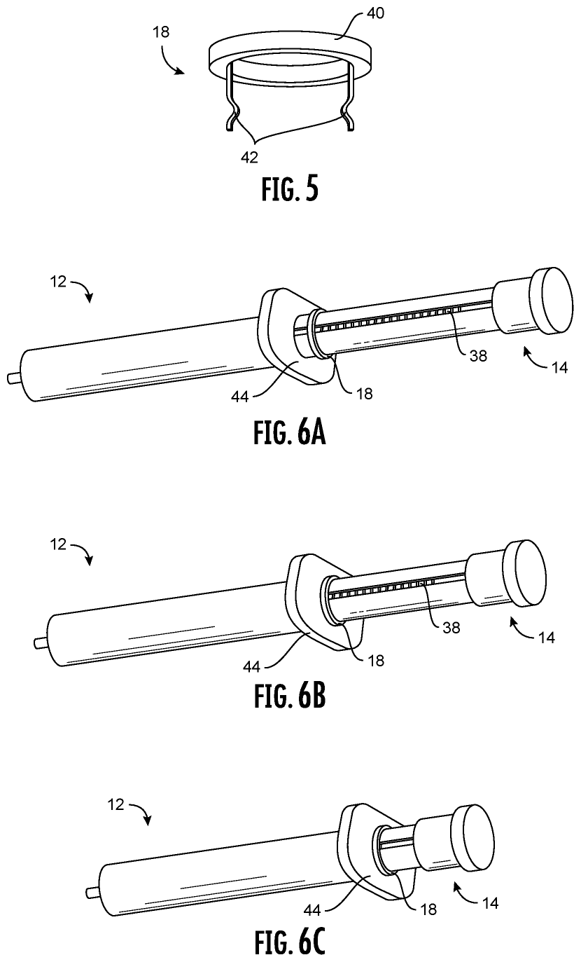

FIG. 2 is a side view of the plunger 14 of the syringe 10 illustrated in FIG. 1. FIG. 3 is an exploded perspective view of the plunger 14. FIG. 4 is a perspective view of the printed circuit board 16 of the plunger illustrated in FIG. 3. FIG. 5 is a perspective view of the conductive ring 18 of the syringe 10 illustrated in FIG. 1. FIGS. 2-5 will be discussed together. As illustrated, the plunger 14 can include a first body portion 22 and a second body portion 24 with the printed circuit board 16 positioned between the first body portion 22 and the second body portion 24. Specifically, the first body portion 22 and the second body portion 24 can each be elongated axially extending halves of the plunger 14 that are coupled together to form the overall plunger 14. Additionally, the printed circuit board 16 can be disposed centrally between the first body portion 22 and the second body portion 24, such that the printed circuit board 16 is positioned radially between outer surfaces of the plunger 14 with respect to the central axis CA of the plunger 14. In other words, the printed circuit board 16 is positioned generally aligned with the central axis CA of the plunger 14, between the first body portion 22 and the second body portion 24, discussed further below.

The first body portion 22 includes a first end 22A and a second end 22B that are positioned at opposite distal ends from each other. Likewise, the second body portion 24 includes a first end 24A and a second end 24B that are positioned at opposite distal ends from each other. The shape and size of the first end 22A of the first body portion 22 is configured to be a mating profile with the first end 24A of the second body portion 24, such that the first ends 22A, 24A can be coupled together. Similarly, the shape and size of the second end 22B of the first body portion 22 is configured to be a mating profile with the second end 24B of the second body portion 24, such that the second ends 22B, 24B can be coupled together to form the overall plunger 14. In some examples, as illustrated, the first end 22A of the first body portion 22 can include a straight portion and the first end 24A of the second body portion 24 can include a hook portion. As such, the first ends 22A, 24A can be coupled together such that the straight portion of the first end 22A is held within the hook portion of the first end 24A. Additionally, the second body portion 24 can include a pin 26 that can be configured to engage with an aperture (not shown) of the first body portion 22 in an interference or friction fit to aid in positioning and coupling the components together. Further, the second ends 22B, 24B can each include mating clip features that are configured to snap-clip together to secure the second ends 22B, 24B together. It is to be understood that the illustrated and disclosed coupling technique is only an example and the first body portion 22 and the second body portion 24 can be coupled together using any other technique that will secure the first body portion 22 and the second body portion 24 together during use of the syringe 10.

Referring again to FIGS. 2-4, the printed circuit board 16 includes a shape that generally corresponds to a shape of the first body portion 22 and the second body portion 24. Specifically, the printed circuit board 16 can generally have a thin and long rectangular shape, such that the printed circuit board 16 fits between the first body portion 22 and the second body portion 24. The printed circuit board 16 can include an aperture 28 extending through a first end 16A of the printed circuit board 16. The aperture 28 can be sized and shaped to accept the pin 26 of the second body portion 24, and the aperture 28 and pin 26 connection is configured to properly position and secure the printed circuit board 16 relative to the first body portion 22 and the second body portion 24. Therefore, to install the printed circuit board 16 to the plunger 14, the first body portion 22 and the second body portion 24 are initially separated (see. FIG. 3) and then the printed circuit board 16 is inserted onto the second body portion 24, such that the pin 26 of the second body portion 24 is inserted into the aperture 28 of the printed circuit board 16. The connection between the aperture 28 of the printed circuit board 16 and the pin 26 of the second body portion 24 properly aligns the printed circuit board 16 on the second body portion 24. Then the first body portion 22 is coupled to the second body portion 24 by coupling the respective first ends 22A, 24A and second ends 22B, 24B together, as previously discussed. Therefore, as illustrated best in FIG. 2, after the printed circuit board 16 is coupled within the plunger 14, the printed circuit board 16 is coupled (i.e., sandwiched) between the first body portion 22 and the second body portion 24 of the plunger 14.

Referring now to FIG. 4, the printed circuit board 16 includes the aperture 28 positioned adjacent the first end 16A of the printed circuit board 16. Further, the printed circuit board 16 includes a battery connector 30 positioned at a second end 16B of the printed circuit board 16, with the second end 16B being an opposite end of the printed circuit board 16 as the first end 16A. The battery connector 30 is an electrical connection feature that is configured to accept a battery 32 (see FIG. 3), which battery 32 provides electrical energy to power and operate the electrical components of the printed circuit board 16. In some examples, as illustrated, the battery 32 can be a coin or button cell battery, and the battery connector 30 can be sized and shaped to secure the battery 32 to the printed circuit board 16. Additionally, the battery connector 30 and the battery 32 can be sized and shaped to fit within an interior of each of the second ends 22B, 24B of the first body portion 22 and the second body portion 24, which protects and seals the battery 32 from encountering fluid or other debris during use. In some examples, as illustrated, a widthwise direction or diameter of the battery 32 can be oriented approximately perpendicular to a direction extending from the first end 16A to the second end 16B of the printed circuit board 16.

The printed circuit board 16 can further include a Bluetooth Low Energy (BLE) chip 34, a ground contact 36, and a plurality of contact pads 38. The BLE chip 34 can also be referred to as a BLE microcontroller or microcontroller, the BLE chip 34 is electrically coupled to the printed circuit board 16, and the BLE chip 34 is configured to receive electrical energy from the battery 32. The BLE chip 34 is configured to read a digital input change of the printed circuit board 16, and then a newly computed status of the syringe 10 can be sent by the BLE chip 34 to a smartphone or other device, which can then store and/or analyze the data received from the BLE chip 34 of the syringe 10, discussed further below. In some examples, the BLE chip 34 can have a transmission range of 10 meters or greater to transfer data to a connected device. The ground contact 36 is positioned and extends along a first lateral side of the printed circuit board 16 from the first end 16A to the second end 16B of the printed circuit board 16. Further, the ground contact 36 is a continuous ground contact 36 that extends fully along a first lateral side of the printed circuit board 16 from the first end 16A to the second end 16B of the printed circuit board 16. The ground contact 36 can be produced from a conductive material, such as for example a copper material.

The plurality of contact pads 38 are positioned and extend along a second lateral side of the printed circuit board 16, opposite the first lateral side, from the first end 16A to the second end 16B of the printed circuit board 16. Further, the plurality of contact pads 38 are a discontinuous group of individual contact pads 38 that are each separated by small nonconductive gaps or distances between each individual contact pad 38. In some examples, as illustrated in FIG. 4, the relative size and shape of each individual contact pad 38 may differ to differentiate between each individual contact pad 38 and the electrical signal produced by each individual contact pad 38. In other examples, as illustrated in FIG. 1, the relative size and shape of each individual contact pad 38 may be the same. In such an example, a different technique may be used to identify each individual contact pad 38, discussed further below. The printed circuit board 16 can be conformal coated in a thin polymeric film that is applied to the printed circuit board 16 in order to protect the printed circuit board 16 and its components from the environment and corrosion. Specifically, the printed circuit board 16 can be conformal coated to improve the resilience to water or other fluid ingress, leaving only the ground contact 36 and the plurality of contact pads 38 exposed for a conductive connection with the conductive ring 18, discussed below.

Referring now to FIG. 5, which is a perspective view of the conductive ring 18 of the syringe 10 illustrated in FIG. 1. The conductive ring 18 includes a circular ring shaped conductive ring body 40 that is constructed from a conductive material, such as for example a copper material. The conductive ring body 40 is shaped to fit around an outer circumference of the plunger 14. As such, the conductive ring body 40 has an inner diameter that is approximately equal to (and slightly larger than) an outer diameter of a least a portion of the plunger 14. Further, the conductive ring body 40 is configured to slide or translate axially along the outer circumference of the plunger 14 during the axial translation of the plunger 14 into the barrel 12. The conductive ring 18 also includes at least one sliding contact 42 coupled to and generally extending axially away from the conductive ring body 40. In the illustrated example, the conductive ring 18 includes two sliding contacts 42. In other examples, the conductive ring 18 can include more or less than two sliding contacts 42. The sliding contacts 42 are configured to engage and contact the ground contact 36 and the plurality of contact pads 38 when the conductive ring 18 is assembled to the plunger 14, discussed below.

FIG. 6A is a perspective view of the syringe 10 in a first position or orientation before an injection. FIG. 6B is a perspective view of the syringe 10 in a second position or orientation during an injection. FIG. 6C is a perspective view of the syringe 10 in a third position or orientation after completing an injection. FIGS. 6A-6C will be discussed together. As illustrated in FIGS. 6A-6C, the conductive ring 18 is coupled to the plunger 14 such that the conductive ring 18 is axially aligned with the central axis CA of the plunger 14. Further, as illustrated in FIG. 6A, the conductive ring 18 is coupled to the plunger 14 such that the conductive ring 18 is positioned near the first ends 22A, 24A, which are inserted into the barrel 12 of the syringe 10. Additionally, as illustrated in FIG. 6A, when the plunger 14 is inserted into the barrel 12 and the barrel 12 is filled with a fluid (e.g. a liquid medication), the conductive ring 18 is axially offset from a barrel flange 44 of the barrel 12. The barrel flange 44 is positioned at an opposite axial end of the barrel 12 as the end in which a needle would be coupled. Further, the barrel flange 44 is a feature of the barrel 12 that a user grasps with their fingers during the injection process of the syringe 10. The barrel flange 44 extends radially outwards from an outer circumference of the barrel 12, with respect to a central axis of the barrel 12, such that the barrel flange 44 has a greater width and/or diameter than an outer surface of the barrel 12.

When the prefilled syringe 10 is opened and removed from its packaging, as illustrated in FIG. 6A, the plunger 14 extends axially out from the end of the barrel 12 including the barrel flange 44. Further, the conductive ring 18 is separated by a gap (i.e., axially offset) from the barrel flange 44. Once the syringe 10 has been primed and the injection is being performed, as illustrated in FIG. 6B, the plunger 14 axially translates into the barrel 12, and the conductive ring 18 translates axially towards the barrel 12 until the conductive ring 18 is pressed against a surface or face of the barrel flange 44. Specifically, the conductive ring 18 translates axially with the plunger 14 until the conductive ring 18 contacts the barrel flange 44, and then the conductive ring 18 remains in contact with the barrel flange 44 until the injection process is completed. Therefore, as long as the plunger 14 moves axially into the barrel 12, the conductive ring 18 is forced to stay in position on the surface or face of the barrel flange 44 due to the mechanical interference with the barrel flange 44.

Further, during the axial insertion or translation of the plunger 14 into the barrel 12, the sliding contacts 42 of the conductive ring 18 are pressed against and slide along the ground contact 36 and the plurality of contact pads 38, which are visible and/or accessible between the first body portion 22 and the second body portion 24 of the plunger 14. The contact and connection between the sliding contacts 42 and the ground contact 36 and the plurality of contact pads 38 produces electrical/digital signals that are transferred to the BLE chip 34 through digital input lines or other conductive connection paths on or within the printed circuit board 16. The BLE chip 34 gathers the data and is able to analyze the data to distinguish the actual position of the plunger 14 relative to the barrel 12, which indicates the status of the injection process. In other words, the electrical connection between the conductive ring 18 and the printed circuit board 16 can be recorded and analyzed by the BLE chip 34 to indicate how far into the injection process the syringe 10 is, and when the injection process of the syringe 10 is complete and the fluid within the syringe 10 has been fully dispensed. Therefore, when the injection is completed, the plunger 14 includes a specific/distinguishable contact pad 38 on the printed circuit board 16 that identifies the final position of the plunger 14 and a proper completion of the injection cycle. As such, the plurality of contact pads 38 are each configured to produce a unique electrical or digital signal that is transferred to the BLE chip 34. Then the BLE chip 34 can analyze the unique electrical or digital signal to determine how far into the injection process the plunger 14 has completed (e.g. initial/start position, halfway through injection process, injection process completed, etc.).

The BLE chip 34 can further be wirelessly connected to a smartphone or other connected device (e.g., a controller, a computer, cloud networking, etc.) and the BLE chip 34 can be configured to transfer the collected data to the connected device for further storing and analysis. As such, the syringe 10 is configured to produce, collect, and record data related to the status of the injection process, and then the syringe 10 is configured to transfer that data from the syringe 10 to a remotely connected device through a wireless transfer protocol. It is also noted that the conductive ring 18 and the plunger 14 are free to rotate about a central axis of the syringe 10 within the barrel 12 since there are no mechanical constraints preventing such rotation. Specifically, the sliding contacts 42 of the conductive ring 18 are positioned within small gaps on either side of the plunger 14 that are created by the connection of the first body portion 22 and the second body portion 24. This connection facilitates the sliding contacts generally being within or flush with an outer diameter of the plunger 14 that is configured to be positioned within the barrel 12. Additionally, the connected conductive ring 18 and the plunger being axially rotatable about an axis of the plunger 14 prevents issues or damage during assembly, which prevents damage and increases ease of use by the user or patient.

The syringe 10 including the printed circuit board 16 within the plunger 14 and the conductive ring 18 provides a digital readout of the injection process and the position of the plunger 14 relative to the barrel 12, which data is useful in clinical trials as well as overall patient monitoring. In some examples, the plurality of contact pads 38 can be configured such that the syringe 10 can identify six positional sensing positions of the plunger 14 relative to the barrel 12 (i.e., initial start position, 50% injection completed, 70% injection completed, 80% injection completed, 90% injection completed, and 100% injection completed). In other examples, the plurality of contact pads 38 can be configured such that the syringe 10 can identify more or less than six positional sensing positions of the plunger 14 relative to the barrel 12. In any case, the data collected can identify the name of the fluid medication, whether a user/patient has administered the correct dosage of the fluid medication, the time and day the fluid medication was dispensed, and/or the speed or duration in which the fluid medication was dispensed. Each of the aforementioned data points can be utilized to analyze the efficacy of the fluid medication and whether changes are required and/or if the patient is correctly administering the fluid medication as instructed.

The syringe 10 including the plunger 14 with the printed circuit board 16 and the conductive ring 18 is an innovative electromechanical switch sensing architecture that is paired with a low power BLE chip 34, which assembly allows the creation of a smart syringe 10 used to produce and transfer injection progress data of the syringe 10 to an application on a smartphone or other connected device. The miniaturization of electronics and the novel position sensor concept disclosed make the body of the syringe 10 compatible with existing thermoformed packages, and the reduced number of components results in a cheaper bill of material and overall syringe 10, compared to previous smart syringes. In some examples, the data can be collected with a medical grade hub or smartphone, and the measurement of the delivered dose can be encrypted for data safety. For at least those reasons, it is clear that the syringe 10 of the present disclosure is advantageous over previous smart syringes.

Additionally, the syringe 10 of the present disclosure provides a cost-effective method to monitor and wirelessly report the position of the plunger 14 of the syringe 10 during the injection process. The plurality of contact pads 38 leveraging a side plating on the printed circuit board 16, and the conductive ring 18 sliding along the plunger 14 is configured to capture the position of the plunger 14 as it is moving axially into the barrel 12 during the injection. This position of the plunger 14 is monitored and analyzed by the BLE chip 34 (i.e., microcontroller) as a digital input change, and a newly computed status is sent via the BLE chip 34 to the smartphone or other connected device on a periodic notification period. Therefore, the smart syringe 10 of the present disclosure can be utilized to automatically gather data relating to the injection process which can be utilized for clinical trials and to enable therapy adherence monitoring.

As will be appreciated by a person having ordinary skill in the art, the smart syringe 10 of the present disclosure can include integrated security protocols to ensure data protection during the collection and transfer of data. The smart syringe 10 can include BLE connectivity to smartphones and other gateways to enable measurements of the delivered dose of medication, and to enable therapy adherence monitoring. Further, the smart syringe 10 can be the same size and dimensions as existing syringes to aid in the ease of use and familiarity of the product, for example the smart syringe 10 can be a 1 mL prefilled syringe in some embodiments. To this point, the smart syringe 10 can be compatible with existing syringe packages to aid in packaging and delivery of the device. Finally, the smart syringe 10 can be produced with an optimized bill of material and a long shelf life (e.g. 4+ years) to create a relatively cheap smart syringe 10 that is also disposable. In some examples, the smart syringe 10 can be produced from eco-friendly resins (e.g. polycarbonates, polypropylene, etc.) such that the smart syringe 10 is at least partially recyclable. The smart syringe 10 of the present application provides many advantages over previous smart syringes, as will be appreciated by those having skill in the art.

Having thus described the present embodiments in detail, it is to be appreciated and will be apparent to those skilled in the art that many physical changes, only a few of which are exemplified in the detailed description of the disclosure, could be made without altering the inventive concepts and principles embodied therein.

It is also to be appreciated that numerous embodiments incorporating only part of the preferred embodiment are possible which do not alter, with respect to those parts, the inventive concepts and principles embodied therein. The present embodiment and optional configurations are therefore to be considered in all respects as exemplary and/or illustrative and not restrictive, the scope of the disclosure being indicated by the appended claims rather than by the foregoing description, and all alternate embodiments and changes to this embodiment which come within the meaning and range of equivalency of said claims are therefore to be embraced therein.

### LOG OF REFERENCE NUMERALS

10 Syringe
12 Barrel
14 Plunger
16 Printed Circuit Board
16A First End
16B Second End
18 Conductive Ring
20 Plunger End
22 First Body Portion
22A First End
22B Second End
24 Second Body Portion
24A First End
24B Second End
26 Pin
28 Aperture
30 Battery Connector
32 Battery
34 BLE Chip
36 Ground Contact
38 Contact Pads
40 Conductive Ring Body
42 Sliding Contacts
44 Barrel Flange
CA Central Axis

What is claimed is:

1. A syringe comprising:
a barrel including a barrel flange positioned at an axial end of the barrel;
a plunger axially translatable within the barrel along a central axis of the plunger, the plunger including a first body portion coupled to a second body portion;
a printed circuit board coupled and positioned between the first body portion and the second body portion, the printed circuit board comprising:
a ground contact disposed on a first lateral side of the printed circuit;
a plurality of contact pads disposed on a second lateral side of the printed circuit board; and
a microcontroller coupled to a surface of the printed circuit board; and
a conductive ring disposed about an outer circumference of the plunger, the conductive ring including sliding contacts configured to engage the ground contact and the plurality of contact pads of the printed circuit board;
wherein the microcontroller is configured to detect a change in conductivity to identify a position of the plunger relative to the barrel.

2. The syringe of claim 1, wherein the first body portion and the second body portion are coupled together through a snap-fit connection.

3. The syringe of claim 1, wherein the first body portion and the second body portion are a first half portion and a second half portion of the plunger, respectively.

4. The syringe of claim 1, wherein the second body portion includes a pin positioned on a radially inner surface of the second body portion, relative to the central axis of the plunger.

5. The syringe of claim 4, wherein the printed circuit board includes an aperture extending through the printed circuit board, the aperture being configured to accept the pin of the second body portion for positioning of the printed circuit board relative to the second body portion.

6. The syringe of claim 1, wherein the printed circuit board includes a battery connector disposed at an end of the printed circuit board, the battery connector being configured for accepting and securing a battery of the syringe.

7. The syringe of claim 6, wherein the battery is a coin cell battery.

8. The syringe of claim 1, wherein the ground contact of the printed circuit board is a continuous ground contact that extends from a first end of the printed circuit board to a second end of the printed circuit board.

9. The syringe of claim 1, wherein the plurality of contact pads are a discontinuous group of individual contact pads that extend from a first end of the printed circuit board to a second end of the printed circuit board.

10. The syringe of claim 9, wherein the discontinuous group of individual contact pads includes a nonconductive space positioned between each adjacent contact pad of the plurality of contact pads.

11. The syringe of claim 1, wherein the microcontroller is a Bluetooth low energy chip that is configured to wirelessly transmit data to a device remote from the syringe.

12. The syringe of claim 1, wherein the surface of the printed circuit board the microcontroller is coupled to is perpendicular to the first lateral side and the second lateral side of the printed circuit board.

13. The syringe of claim 1, wherein the first lateral side of the printed circuit board is parallel with the second lateral side of the printed circuit board.

14. The syringe of claim 1, wherein the syringe is a pre-filled disposable syringe.

15. The syringe of claim 1, wherein the conductive ring includes a circular shaped conductive ring body and a first sliding contact and a second sliding contact each extending axially away from the conductive ring body.

16. The syringe of claim 15, wherein the first sliding contact is configured to engage the ground contact of the printed circuit board, and the second sliding contact is configured to engage the plurality of contact pads of the printed circuit board.

17. The syringe of claim 1, wherein the conductive ring includes an inner diameter greater than an outer diameter of a portion of the plunger configured to axially translate into the barrel.

18. The syringe of claim 1, wherein the conductive ring is axially translatable along an outer surface of the plunger during an axial translation of the plunger into the barrel.

19. The syringe of claim 18, wherein during the axial translation of the plunger into the barrel the conductive ring contacts the barrel flange, preventing axial translation of the conductive ring relative to the barrel.

20. The syringe of claim 18, wherein before the axial translation of the plunger into the barrel, the conductive ring is axially offset from the barrel flange.
